(19) United States
(12) Patent Application Publication
(10) Pub. No.: US 2008/0118984 A1
(43) Pub. Date: May 22, 2008

(54) Biosensing system and related biosensing method

(75) Inventors: Wen Ching Yuan, Hsin-Chu City (TW); Murphy Chiu, Hsinchu (TW)

Correspondence Address: BIRCH STEWART KOLASCH & BIRCH, PO BOX 747, FALLS CHURCH, VA, 22040-0747, US

(73) Assignee: EPS Bio Technology Corp., Hsin-Chu City (TW)

(21) Appl. No.: 11/603,185

(22) Filed: Nov. 22, 2006

(51) Int. Cl. G01N 33/48

(52) U.S. Cl. 436/63; 422/68.1

(57) ABSTRACT

A biosensing system for testing a testing strip to generate a testing result includes: a timing module, for determining a time parameter according to a time point when the testing strip is manufactured; and a testing module, for performing a biochemical operation on the testing strip to generate an original testing result; a compensating module, coupled to the testing module and the timing module, for adjusting the original testing result according to the time parameter to generate the testing result.

### BACKGROUND OF THE INVENTION

1. Field of the Invention

The present invention relates to a biosensing system and related biosensing method, and more particularly, to a biosensing system and related biosensing method having warning mechanism and compensating mechanism.

2. Description of the Related Art

In general, a biosensing device is utilized to measure the concentration of biological materials (for example, blood glucose concentration) in humans' sample (such as a blood sample). Therefore, a diabetic can monitor his blood glucose concentration by using the biosensing device to maintain his health.

U.S. Pat. No. 5,366,609 discloses a biosensing device, which utilizes a read-only-memory key (ROM key) to select a specific biosensing procedure to be executed or to provide parameters for biosensing procedures to achieve the purpose of measuring the blood samples. The biosensing device (meter) employs disposable sample strips (testing strips) that include a reaction region for receiving a blood sample. When the blood sample is being tested, a bio-chemical reaction is triggered in the reaction region such that a testing result can be obtained. Furthermore, when different testing strips (testing samples) are plugged into the biosensing device, different ROM keys should be used to inform the biosensing device to execute a corresponding biosensing procedure according to different testing strips or to execute the biosensing procedure according to different parameters.

In general, the testing strips contain some biological materials, such as proteins, which can react with the blood sample to generate the testing result. But, the properties of the biological materials change as time goes by. This causes often introduces an error of the testing result. In this way, if the error of the testing result is large enough, the testing result is no longer reliable.

In order to prevent from the above-mentioned situation, an expiration date is often provided. In general, the expiration date is often set according to the error of testing result (for example, it can be set when the error reaches 10%). The expiration date is utilized to remind the user not to use expired testing strips. For example, the expiration date can be labeled on the vial of the testing strips to inform the users. Or, the biosensing device can inform the user through the display device.

From the above disclosure, it can be seen that the only way we deal with the expired testing strip is “not to use it”. The user has to buy new testing strips, and therefore spend more money.

Furthermore, nobody can ensure that the time stored in the biosensing meter is correct. In the general case, the time stored in the biosensing meter is reset when the electricity runs out. In this case, the user has to input the current time into the biosensing meter when the battery is changed. Therefore, the time stored inside the biosensing meter cannot be trusted such that the untrustful time cannot be utilized to do further things, such as compensation.

### SUMMARY OF THE INVENTION

In view of the above-mentioned problems, an object of the invention is to provide a biosensing system capable of compensating the testing result according to the manufacturing date of the testing strip.

According to an embodiment of the present invention, a biosensing system for testing a testing strip to generate a testing result is disclosed. The biosensing system comprises: a timing module, for determining a time parameter according to a time point when the testing strip is manufactured; and a testing module, for performing a bio-chemical operation on the testing strip to generate an original testing result; a compensating module, coupled to the testing module and the timing module, for adjusting the original testing result according to the time parameter to generate the testing result.

According to another embodiment of the present invention, a biosensing method for testing a testing strip to generate a testing result is disclosed. The biosensing method comprises: generating a time parameter according to a time point when the testing strip is manufactured; performing a biochemical operation on the testing strip to generate an original testing result; and adjusting the original testing result according to the time parameter to generate the testing result.

According to an embodiment of the present invention, a warning system utilized in a biosensing system is disclosed. The warning system comprises: an environment parameter detecting module, for detecting whether an environment parameter of environment of the testing strip complies with a predetermined condition; and a warning module, coupled to the environment parameter, for generating warning information if the environment parameter complies with the predetermined condition.

According to another embodiment of the present invention, a warning method utilized in a biosensing system is disclosed. The warning method comprises: detecting whether an environment parameter of environment of the testing strip complies with a predetermined condition; and generating warning information if the environment parameter complies with the predetermined condition.

The present invention comprises a compensating mechanism and a warning mechanism such that the testing strip can be used for a longer time and the biosensing device can inform the user if the biological material of the testing strip 120 may be ruined due to the environment

### BRIEF DESCRIPTION OF THE DRAWINGS

FIG. 1 is a functional block diagram of a biosensing system according to the present invention.

FIG. 2 is a diagram illustrating the relationships between errors of the testing results and the time since the testing strip has been manufactured.

FIG. 3 is a diagram of an embodiment of the detecting module shown in FIG. 1.

### DETAILED DESCRIPTION OF THE INVENTION

The “TITLE” of the invention will be described with reference to the accompanying drawings.

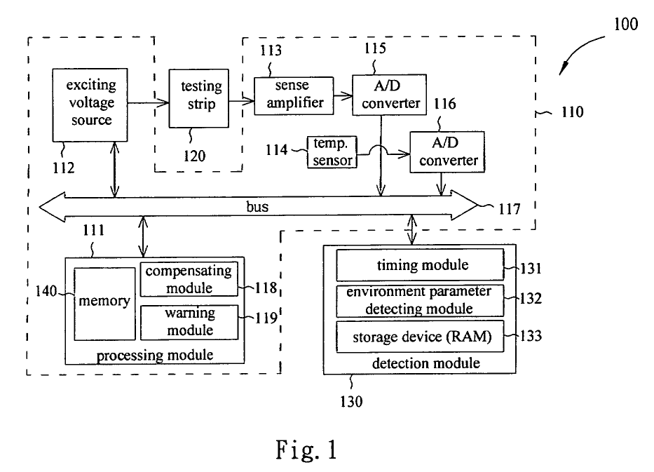

Please refer to FIG. 1, which is a functional block diagram of a biosensing system 100 according to the present invention. As shown in FIG. 1, the biosensing system comprises a biosensing device 110, and testing strip 120, and a detection module 130. Basically, the biosensing device 110 is utilized for triggering a biochemical reaction on the testing strip to measure the testing strip 120 such that an original testing result is generated.

In this embodiment, the biosensing device 110 comprises a processing module 111, an exciting voltage source 112, a sense amplifier 113, a temperature sensor 114, two A/D converters 115 and 116, and a bus 117. Please note, the electrical connections among these components are shown in FIG. 1, and further illustrations are omitted here.

The testing operation is quite similar to that disclosed U.S. Pat. No. 5,366,609. For example, the exciting voltage source 112 provides an exciting voltage to the testing strip 120 such that a bio-chemical reaction is performed. And then, the sense amplifier 113 obtains the reaction result from the testing strip 120, and the A/D converter 115 transforms the reaction result into a digital reaction result.

On the other hand, the temperature sensor 114 detects the environment temperature when the bio-chemical reaction takes places, and the A/D converter 116 transforms the detected temperature into a digital temperature value.

And then, the processing module 111 obtains the digital reaction result and the digital temperature value through the bus 117, and calculates an original testing result according to the digital reaction result and the digital temperature value

As mentioned previously, in the prior art, the original testing result is regarded as a final testing result, which is reported to the user through a display device (not shown). But, as mentioned previously, the original testing result may not be reliable due to the variance of the biological materials of the testing strip 120.

Therefore, the present invention further comprises a compensating mechanism (including the compensating module 118 and the detection module 130, which will be illustrated in the following disclosure) for compensating the variance of the testing strip 120 such that a more correct testing result can be provided to the user.

The compensating technique of the present invention is illustrated as follows. First of all, as is known, we can find a corresponding relationship between measured testing results and the manufacturing date for each testing strip. Therefore, the measured testing result can be adjusted accordingly to generate a correct testing result.

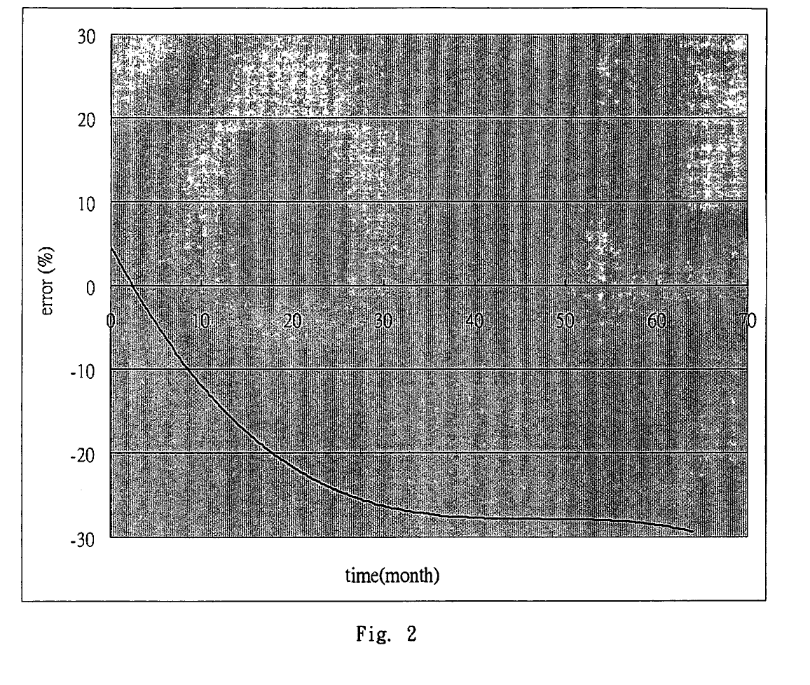

Please refer to FIG. 2, which is a diagram illustrating the relationships between errors of the testing results and the time since the testing strip has been manufactured. In FIG. 2, the horizontal axis represents the time since the testing strip has been manufactured, and the vertical axis represents the errors of the measured testing result. As shown in FIG. 2, for each time point, a corresponding error can be found according to the curve. For example, if the testing strip has been manufactured for ten months, the error of the measured testing result should be about 10%. Therefore, the measured testing result needs to be adjusted by 10%.

Furthermore, the compensation method is not hard for those skilled in the art. For example, if time is regarded as an independent variable, a function corresponding to the curve can be determined, for instance, as E(t)=−0.0003 t³+0.038 t²−1.6458 t+5.2764.

And then, if the time (for example, from the testing strip is manufactured to the testing strip is being tested) can be determined, it is easily to find the error E(t) of the original result Rₒᵣᵢ.

Now, the original result Rₒᵣᵢ and the error E(t) are both derived. The compensation result R_{comp} can be determined from the following equation:

R_{comp}=Rₒᵣᵢ/[(100+E(t)]/100] Please note, the above-mentioned curve shown in FIG. 2 can be an experimental result, and the equation and the function can be previously determined and stored in the biosensing device 110 (such as the memory 140 of the processing module 111) for compensation. In this way, the present invention can compensate the original testing result. In the actual implementation, if the error of the original result is about 30%, but after the present invention compensation, the error can be reduced to 6%.

Please refer to FIG. 1 again, the processing module 111 comprises a compensating module 118 and a warning module 119, and the detection module 130 comprises a timing module. 131, an environment parameter detecting module 132, and a storage device 133. The compensating module 118 and the timing module 131 are utilized to perform the above-mentioned compensation. Furthermore, the environment parameter detecting module 132 and the warning module 119 are utilized to inform the user not to the testing strip 120 because the testing strip 120 may be ruined.

The timing module 131, as its name implies, is utilized to time from a specific time point of the testing strip 120. For example, the timing module 131 can start timing when the testing strip 120 is manufactured such that a time parameter can be obtained. And then, the timing module 131 stores the time parameter into the storage device 133. Therefore, the compensating module 118 can obtain the time parameter from the storage device 133 through the bus 117, and compensates the original testing result to generate a correct testing result according to the time parameter and the above-mentioned curve (the relationship between the errors and the time parameter) stored in the memory 140. As mentioned previously, the compensating module 118 can utilize the above-mentioned equation and the function, which are previously stored in the memory 140, and substitute the time parameter and the original testing result into the equations such that the compensation result is derived.

In this way, the testing strip 120 can also be utilized even if the testing strip 120 is expired. In other words, the errors caused by the time factor can be removed/reduced.

Unfortunately, in addition to the time factor, there is still another factor capable of ruining the biological material of the testing strip 120. For example, the testing strip 120 needs to be stored in a specific temperature range. If the environment temperature is too high/low, the biological material of the testing strip 120 may be ruined.

Therefore, the environment parameter detecting module 132 is utilized to continuously detect the environment parameter to see whether if the environment parameter complies with a predetermined condition. And the processing module

In this embodiment, the environment parameter detecting module 132 detects the environment temperature of the testing strip 120. If the temperature has ever been higher than a predetermined value or lower than another predetermined value, the environment parameter detecting module 132 stores a value (such as 1) inside the storage device 133. Otherwise, the value stored inside the storage device 133 does not correspond to 0.

Therefore, the warning module 119 inside the processing module 110 can obtain the value from the storage device 133 through the bus 117. If the value corresponds to 1, the warning module 119 generates warning information to inform the user not to use the testing strip 120 because the testing strip 120 may be ruined.

For example, the warning module 119 can utilize a display device (not shown in FIG. 1) to show warning information to the user.

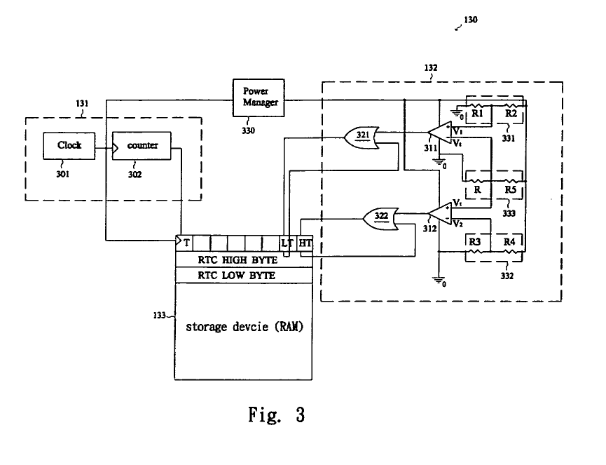

Please refer to FIG. 3, which is a diagram of an embodiment of the detecting module 130 shown in FIG. 1. As shown in FIG. 3, the timing module 131 comprises a clock signal generator 301 and a counter 302, and the environment parameter detecting module comprises a plurality of comparators 311 and 312, a plurality of OR gates 321 and 322, and a plurality of voltage divider circuit 331, 332, and 333. In addition, the storage device 133 is implemented by a random access memory (RAM).

Furthermore, because the RAM 133 is a volatile memory, it means that the RAM 133 should be continuously powered such that the data stored inside the RAM 133 can be maintained. Therefore, the detecting module 130 comprises a power manager 330 to supply power to the RAM 133 and the other devices (including the above-mentioned clock, counter, logic gates, and comparators). Please note, the electrical connections among the above-mentioned devices are shown in FIG. 3, and thus omitted here.

The operation and function of the timing module 131 are illustrated as follows. In this embodiment, the counter 302 is utilized to count according to the clock signal outputted by the clock signal generator 301. For instance, the counter 302 can be triggered by the rising/falling edges of the clock signal such that a count value can be generated.

Therefore, if the clock generator 301 is set to start generating the clock signal from a specific time point (such as the time point that the testing strip is manufactured), the count value can represent the time period (regarded as a time parameter) from the specific time point to the current time. Furthermore, the count value is then stored inside the RAM 133 (as is shown, in this embodiment, the high byte part of the count value can be stored inside the “RTC high byte” of the RAM 133, and the low byte part of the count value can be stored inside the “RTC low byte” of the RAM 133). As mentioned previously, the compensating module 118 of the processing module 111 then utilizes the count value and the relationship curve stored inside the memory 140 to compensate the original testing result.

Please note, the above-mentioned counter 302 is utilized to calculate the time interval between the current time and the time point when the testing strip is manufactured. As mentioned previously, this is because the time information stored inside the prior art biosensing device cannot be trusted.

However, in many countries (such as Germany), a radio clock signal (such as DCF77 in Germany) has been developed to provide real time information. The time information, for example, often corresponds to standard time information, which can be utilized for time synchronization.

As is known, for real-time systems (such as traffic real-time monitering system, network synchronization system, weather monitering system, GPS), all components of these systems need to be synchronized by the same time information. Therefore, the radio clock signal provide the real time information to the above-mentioned systems.

Since the real time information is reliable, in another embodiment of the present invention, the timing module 131 can be implemented by a radio clock receiver, which is used to receive a radio clock signal and generate real time information according to the radio clock. In other words, the radio clock receiver can replace the above-mentioned counter.

Furthermore, the implementation of the above-mentioned structure is not hard for those skilled in the art. For example, in order to perform the compensation operation, the compensating module 118 needs to know the time interval between the current time and the time point when the testing strip is manufactured. Since the current time can be provided by the radio clock receiver, as long as the storage device 133 stores the time point when the testing strip is manufactured, the compensating module 118 can easily the time interval, and then compensate the original testing result according to the time interval and the relationship curve.

On the other hand, the operation and function of the environment parameter detecting module 132 are illustrated as follows. The voltage dividing circuit 331 is utilized to provide a voltage level V1 according to the resistances of the resistors R1 and R2. Similarly, the voltage dividing circuit 332 provides a voltage level V2 according to the resistances of the resistors R3 and R4. Please note, the resistances of the resistors R1, R2, R3, and R4 are predetermined for providing appropriate voltage levels VI and V2.

On the other hand, the voltage dividing circuit 333 comprises a resistor R5 and a thermal resistor R. The resistance of the thermal resistor R is dependant to the temperature. Therefore, the voltage level Vt provided by the voltage dividing circuit 333 is also temperature-dependant.

In this embodiment, the environment parameter detecting module 132 is utilized to continuously measure the environment temperature of the testing strip 120 to see whether the testing strip 120 has encountered an extreme temperature which may ruin the testing strip 120.

Therefore, the voltage levels V1 and V2 should be properly set to reflect the temperature thresholds of the testing strip 120 such that the environment parameter detecting module 132 can work correctly.

And then, the comparators 311 and 312 respectively compare the voltage level Vt with voltage levels V1 and V2, and output a logic value according to the comparison results. For example, the comparators 311 and 312 can be designed to detect whether the voltage level Vt is the same as the voltage levels V1 and V2. And if the voltage level Vt is the same as the voltage level V1, the comparator 311 outputs the logic level 1. On the other hand, the comparator 312 outputs the logic level 1 if the voltage level Vt is the same as the voltage level V2.

And then, the OR gate 321 performs an OR logic operation on the output of the comparator 311 and a value stored inside a register LT of the RAM 133 and output a logic value to the register LT. Because the value stored in the LT is an input of the OR gate 311, if the output of the comparator 311 has ever been equal to logic level 1, the output of OR gate 321 and the value stored inside the register LT are both fixed as the logic level 1.

Similarly, if the output of the comparator 312 has ever been equal to logic level 1, the output of OR gate 322 and the value stored inside the register HT are both fixed as the logic level 1.

From the above disclosure, it can be easily seen that the values stored inside the registers LT and HT can be indicators, which indicates whether the environment temperature has ever been higher than a predetermined threshold or lower than another predetermined threshold.

As mentioned previously, the warning module 119 of the processing module 111 can read the values stored inside the registers LT and HT from the RAM 133 through the bus 117. Therefore, the warning module 119 can generate warning information if each of the values stored inside the registers LT and HT corresponds to 1. And the warning information can be shown, for instance in a display device, to inform the user not to use the testing strip 120.

Please note, in the aforementioned embodiment, the storage device 133 is implemented by a random access memory. But in the actual implementation, the storage device 133 can be implemented by all kinds of writable memory devices, including volatile and non-volatile memory devices. This change also obeys the spirit of the present invention.

Furthermore, the storage space of the storage device 133 is not limited. In addition to the values and the count value, the storage device 133 can store more data, such as program parameters or information of the testing strip 120. That is, the RAM 133 can not only be utilized for the above-mentioned compensation and warning mechanism of the present invention, but also provide the function of the ROM key of the prior art.

In addition, it can be seen that the detection device 130 can detect some properties of the testing strip 120. So, the detection device 130 should be placed near the testing strip 120.

Therefore, the detection device 130 can be a pluggable device, or only the storage device 133 can be a pluggable device. In this way, when the testing strip 120 should be measured, the detection device 130 (or the storage device 133) is plugged in.

Moreover, although in the above-mentioned embodiment, the environment parameter detecting module 132 is utilized to compare the environment temperature with temperature thresholds. But in the actual implementation, the environment parameter detecting module 132 can be designed to detect other environment parameters (such as humidity), which may influence the testing strip 120. Furthermore, the warning module should be designed to inform the user according to the additional environment parameters.

In contrast to the prior art, the present invention comprises a compensating mechanism and a warning mechanism such that the testing strip can be used for a longer time and the biosensing device can inform the user if the biological material of the testing strip 120 may be ruined due to the environment.

While certain exemplary embodiments have been described and shown in the accompanying drawings, it is to be understood that such embodiments are merely illustrative of and not restrictive on the broad invention, and that this invention should not be limited to the specific construction and arrangement shown and described, since various other modifications may occur to those ordinarily skilled in the art.

What is claimed is:

1. A biosensing system for testing a testing strip to generate a testing result, the biosensing system comprising:
a timing module, for determining a time parameter; and
a biosensing device, for performing a bio-chemical reaction on the testing strip to generate an original testing result;
a compensating module, coupled to the testing module and the timing module, for adjusting the original testing result according to the time parameter to generate the testing result.

2. The biosensing system of claim 1, wherein the timing module comprises:
a counter, for generating a count value as the time parameter according to the time point when the testing strip is manufactured; and
a storage module, coupled to the counter, for storing the count value.

3. The biosensing system of claim 2, wherein the counter starts to count from the time point when the testing strip is manufactured.

4. The biosensing system of claim 2, wherein the storage module comprises:
a volatile memory, for storing the count value;
a power supply, for providing a power to the volatile memory to prevent the volatile memory from losing the count value.

5. The biosensing system of claim 4, wherein the volatile memory is a random access memory.

6. The biosensing system of claim 2, wherein the storage module comprises:
a non-volatile memory, for storing the count value

7. The biosensing system of claim 1, wherein the testing module and the compensating module are both embedded in a biosensing device.

8. The biosensing system of claim 1, further comprising:
an environment parameter detecting module, for detecting whether an environment parameter of environment of the testing strip complies with a predetermined condition; and
a warning module, coupled to the environment parameter, for generating warning information if the environment parameter complies with the predetermined condition.

9. The biosensing system of claim 8 wherein the environment parameter detecting module comprises:
a storage device; and
a detecting device, for detecting whether an environment parameter of environment of the testing strip complies with a predetermined condition and storing a predetermined value in the storage device if the environment parameter complies with the predetermined condition;
wherein if the warning module reads the predetermined value from the storage device, the warning module generates warning information.

10. The biosensing system of claim 9, wherein the environment parameter is a temperature degree of surrounding environment of the testing strip, and if the temperature degree has ever been higher than a first predetermined value or less than a second predetermined value, the detecting device determines that the environment parameter complies with the predetermined condition.

11. The biosensing system of claim 8, wherein the testing module, the compensating module, and the warning module are all embedded in a biosensing device.

12. The biosensing system of claim 1, wherein the timing module comprises:
a radio clock receiver, for receiving a radio clock signal and generating real time information as the time parameter according to the radio clock signal; and
a storage module, for storing a time point when the testing strip is manufactured;
wherein the compensating module adjusts the original testing result according to the time parameter and the time point to generate the testing result.

13. A biosensing method for testing a testing strip to generate a testing result, the biosensing method comprising:
generating a time parameter according to a time point when the testing strip is manufactured;
performing a bio-chemical operation on the testing strip to generate an original testing result; and
adjusting the original testing result according to the time parameter to generate the testing result.

14. The biosensing method of claim 12, wherein the step of generating a time parameter according to the time point when the testing strip is manufactured comprises:
starting timing from the time point when the testing strip is manufactured to generate the time parameter.

15. The biosensing method of claim 12, further comprising:
detecting whether an environment parameter of environment of the testing strip complies with a predetermined condition; and
generating warning information if the environment parameter complies with a predetermined condition.

16. The biosensing method of claim 15, wherein the environment parameter is a temperature degree of surrounding environment of the testing strip, and if the temperature degree has ever been higher than a first predetermined value or less than a second predetermined value, the environment parameter complies with the predetermined condition.

17. The biosensing method of claim 11, wherein the timing module comprises:
generating real time information as the time parameter according to a radio clock signal; and
adjusting the original testing result according to the time parameter and the time point to generate the testing result.

18. A warning system utilized in a biosensing system, the warning system comprising:
an environment parameter detecting module, for detecting whether an environment parameter of environment of the testing strip complies with a predetermined condition; and
a warning module, coupled to the environment parameter, for generating warning information if the environment parameter complies with the predetermined condition.

19. The biosensing system of claim 18, wherein the environment parameter detecting module comprises:
a storage device; and
a detecting device, for detecting whether an environment parameter of environment of the testing strip complies with the predetermined condition and storing a predetermined value in the storage device if the environment parameter complies with the predetermined condition;
wherein if the warning module reads the predetermined value from the storage device, the warning module generates warning information.

20. The biosensing system of claim 19, wherein the environment parameter is a temperature degree of surrounding environment of the testing strip, and if the temperature degree has ever been higher than a first predetermined value or less than a second predetermined value, the detecting device determines that the environment parameter complies with the predetermined condition.

21. A warning method utilized in a biosensing system, the warning method comprising:
detecting whether an environment parameter of environment of the testing strip complies with a predetermined condition; and
generating warning information if the environment parameter complies with the predetermined condition.

22. The method of claim 21, wherein the environment parameter is a temperature degree of surrounding environment of the testing strip, and if the temperature degree has ever been higher than a first predetermined value or less than a second predetermined value, the environment parameter complies with the predetermined condition.
